(19) United States
(12) Patent Application Publication
(10) Pub. No.: US 2005/0197683 A1
(43) Pub. Date: Sep. 8, 2005

(54) MICROTUBES FOR SURGERY AND DENTISTRY

(76) Inventor: Samuel R. Levatino, Baton Rouge, LA (US)

Correspondence Address: REGINALD F ROBERTS JR, PO BOX 4535, BATON ROUGE, LA, 70821-4535, US

(21) Appl. No.: 10/908,761

(22) Filed: May 25, 2005

Related U.S. Application Data
(63) Continuation-in-part of application No. 10/605,352, filed on Sep. 24, 2003.

(51) Int. Cl. A61N 1/00

(52) U.S. Cl. 607/89

(57) ABSTRACT

A microtube for surgery and dentistry, and a method for using the microtube in a surgical or dental procedure. The method involves (a) providing a microtube having anterior and posterior ends, a side port, an axial opening at the posterior end of the tubular microtube, and an inner optical core of a material capable of transmitting, guiding, and directing a laser beam, the inner optical core extending from the axial opening at the posterior end of the microtube through the side port; (b) positioning the side port of the microtube at the site of the surgical or dental procedure; (c) placing the axial opening at the posterior end of the tubular member at a source of the laser beam; and (d) delivering the laser beam through the side port to the site of the surgical or dental procedure, thus combining mechanical and chemical debridement into a single procedure, enabling removal of pulpal tissue in three-dimensional volume elements which files and other instruments cannot reach, sterilizing the site of the surgical or dental procedure, and ablating the pulpal tissue.

### CROSS-REFERENCE TO RELATED APPLICATION

This application is a continuation-in-part of application Ser. No. 10/605,352, filed Sep. 24, 2003.

### BACKGROUND OF THE INVENTION

The present invention relates to the fields of medicine and dentistry. More particularly, the invention relates to microtubes for surgical and dental procedures.

### SUMMARY OF THE INVENTION

In general, the present invention in a first aspect provides a microtube for surgery and dentistry. A first embodiment of the microtube comprises a tubular member having anterior and posterior ends; an interior axial opening extending from the anterior to the posterior end of the tubular member; and a side port constructed and arranged for connecting the axial opening to a site of a surgical or dental procedure, and for delivery to the site of a therapeutic agent to be used for the surgical or dental procedure.

A second embodiment of the microtube comprises a tubular member having anterior and posterior ends, a side port, an axial opening at the posterior end of the tubular member, and an inner optical core of a material capable of transmitting, guiding, and directing a laser beam, the inner optical core extending from the axial opening at the posterior end of the tubular member through the side port.

In a second aspect, the invention provides a method for transmitting a therapeutic agent to a site of a surgical or dental procedure. The method comprises (a) providing a microtube comprising a tubular member having anterior and posterior ends; an interior axial opening extending from the anterior to the posterior end of the tubular member; and a port constructed and arranged for connecting the axial opening to the site of the surgical or dental procedure, and for delivery to the site of the therapeutic agent to be used for the surgical or dental procedure; (b) connecting the port of the tubular member to the site of the surgical or dental procedure; (c) connecting the axial opening at the posterior end of the tubular member to the source of the therapeutic agent; and (d) delivering the therapeutic agent to the site of the surgical or dental procedure.

In a third aspect, the invention provides a method for a surgical or dental procedure utilizing laser technology. The method comprises (a) providing a microtube comprising a tubular member having anterior and posterior ends, a side port, an axial opening at the posterior end of the tubular member, and an inner optical core of a material capable of transmitting, guiding, and directing a laser beam, the inner optical core extending from the axial opening at the posterior end of the tubular member through the side port; (b) disposing the side port of the tubular member at the site of the surgical or dental procedure; (c) disposing the axial opening at the posterior end of the tubular member at a source of the laser beam; and (d) delivering the laser beam through the side port to the site of the surgical or dental procedure, thereby combining mechanical and chemical debridement into a single procedure, enabling removal of pulpal tissue in three-dimensional volume elements which files and other instruments cannot reach, sterilizing the site of the surgical or dental procedure, and ablating the pulpal tissue.

### BRIEF DESCRIPTION OF THE DRAWINGS

FIG. 1 is a schematic cross-sectional representation of a first embodiment of a microtube for surgery and dentistry, and an auxiliary member, made in accordance with the principles of the present invention.

FIG. 2 is a schematic cross-sectional representation of a second embodiment of a microtube for surgery and dentistry, and an auxiliary member, made in accordance with the principles of the present invention.

### DETAILED DESCRIPTION OF THE INVENTION

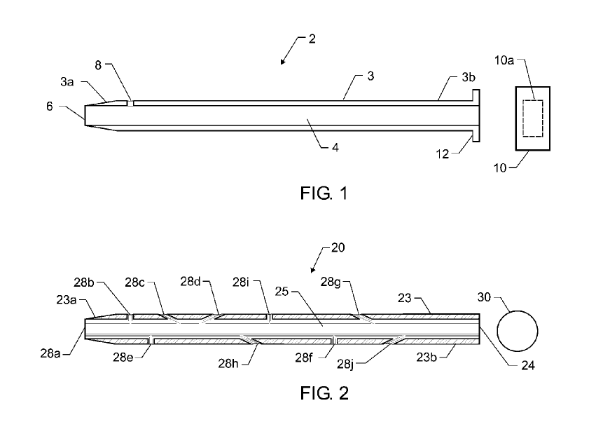

More specifically, reference is made to FIG. 1, in which is shown a first embodiment of a microtube for surgery and dentistry, made in accordance with the principles of the present invention, and generally designated by the numeral 2.

The microtube 2 comprises a tubular member 3 having anterior and posterior ends 3a and 3b, respectively; an interior axial opening 4; and front and side ports 6 and 8, respectively, constructed and arranged for connecting the axial opening to a site of a surgical or dental procedure, and for delivery to the site of a therapeutic agent 10a being used for the surgical or dental procedure. Instead of a single axial opening a plurality of openings may be utilized, and are often beneficial. The posterior end 3b of the tubular member 3 is provided with means 12 for connecting the microtube 2 to a source 10 of the therapeutic agent 10a.

During the surgical or dental procedure, the microtube 2 is connected to the source 10 of the therapeutic agent 10a, which is generally pressure, vacuum, or a pharmaceutical agent, and the therapeutic agent 10a is transmitted through the microtube 2 to the site of the surgical or dental procedure via the axial opening 4 and one or both of the ports 6 and/or 8. The pharmaceutical agent is usually an antibiotic, a chemotherapeutic agent, or a sealant.

Because of its microsize, the microtube 2 does not harm tissue when used to transmit pressure, vacuum, or pharmaceuticals to the site being operated upon, and is especially suited for surgical and dental procedures such as a root canal and surgical operations involving extremely limited space.

The wall of the tubular member 3 can be made of any material compatible with surgical and dental use, and can be of any thickness commensurate with a microtube. Preferably, however, the tubular member 3 is made of metal, and is fabricated by metallic electrodeposition. The anterior end 3a of the tubular member 3 can be cylindrical or tapered. If, however, the tubular member 3 is to be used to perform a root canal, the anterior end 3a is necessarily tapered as shown in FIG. 1; viz., conically tapered from wide to narrow in a direction away from the posterior end 3b toward the anterior end 3a of the tubular member 3. Any one of several types and sizes of taper are utilizable.

Because of the provision of front and side ports, the microtube 2 provides versatility in enabling the surgeon, dentist, or oral surgeon to reach various areas of limited accessibility at the site of the surgical or dental procedure. The combination of multiple ports disposed perpendicularly to one another, and of the extremely small dimensions of the microtube 2, provides a unique instrument for surgeons, dentists, and oral surgeons performing operations in a space limited both in area and in accessibility.

Reference is now made to FIG. 2, in which is shown a second embodiment of a microtube for surgery and dentistry, made in accordance with the principles of the present invention, and generally designated by the numeral 20.

The microtube 20 comprises a tubular member 23 having anterior and posterior ends 23a and 23b, respectively; a posterior axial opening 24; a front port 28a; side ports 28b-28j; and an inner optical core 25 of a material capable of transmitting, guiding, and directing a laser beam from a source 30, the inner optical core 25 extending from the axial opening 24 at the posterior end 23b through the ports 28a-28j.

The wall of the tubular member 23 can be made of any material compatible with surgical and dental use, and can be of any thickness commensurate with a microtube. It may even be a hardened shell of the material comprising the optical core 25. Preferably, however, the tubular member 23 is made of metal, and is fabricated by metallic electrodeposition.

The anterior end 23a of the tubular member 23 can be cylindrical or tapered. If, however, the tubular member 23 is to be used to perform a root canal, the anterior end 23a is necessarily tapered as shown in FIG. 2; viz., conically tapered from wide to narrow in a direction away from the posterior end 23b toward the anterior end 23a of the tubular member 23. Any one of several types and sizes of taper are utilizable.

The side ports 28b-28j provide extreme versatility by virtue of their being capable, by rotation of the microtube 20 about its longtitudinal axis, of disposing the ports 28a-28j in an axial perimeter of at least about one hundred and sixty degrees and a rotational perimeter of a full three hundred and sixty degrees, by disposing the side ports 28b-28j in perpendicular, oblique, and obtuse orientations with reference to the axial front port 28a. Different arrangements of portal openings may be indicated to achieve different surgical or dental objectives.

Light waves normally travel in straight lines. In order to change the direction of a light beam, it is necessary to provide means such as an optical core of a material capable of transmitting, guiding, and directing the light beam. The optical core 25 transmits, guides, and directs the laser beam through the ports 28a-28j. In the absence of such an optical core, the laser beam would be transmitted through only the axial port 28a. The material most generally used to guide and direct a laser beam is an optical fiber. See, for example, Jeff Hecht, Understanding Lasers. Indianapolis, Ind.: Howard W. Sams & Company, 1988, p. 152.

Means other than an optical core can and may be used to transmit, guide, and direct the laser beam. Such means include, but are not limited to, mirrors, reflectors, and reflecting surfaces. Generally, any means capable of transmitting, guiding, and directing a beam of light can and may be used to transmit, guide, and direct a laser beam through the side ports 28b-28j. The critical point is that, in the absence of such means, the laser beam could not be transmitted through the side ports.

Because both the focal length and the wavelength of the laser beam are adjustable, the beam is capable of ablating diseased tissue in conformity with the focal length that is set. This can be done without harm to non-diseased, healthy tissue in very hard-to-reach, relatively inaccessible areas. Such areas cannot be reached or accessed with and by an instrument as small as the smallest needle probe. This microtube laser technology enables early surgery, limited only by early diagnosis.

A particularly important application of the present invention is the provision of an improved procedure for doing a root canal. Use of the microtube 20 as a side-firing laser tube combines mechanical canal debridement and chemical canal debridement into a single procedure. It is presently virtually impossible to clean thoroughly a root-canal system with instruments alone. The use of chemical debridement enables removal of vital and/or nonvital pulpal tissue in three-dimensional volume elements which files and conventional instruments cannot reach. A couple of passes of the side-firing laser tube 20 to the apex of the tooth will clean, debride, and sterilize the site to a greater extent than any existing combination of state-of-the-art procedures. The side-firing laser tube 20 is so small that it will go all the way down to the apex of the tooth without the need for canal enlargement. It will then sterilize the canal and ablate all pulpal tissue.

While certain embodiments and details have been described to illustrate the principles of the present invention, it will be apparent to those skilled in the art that many modifications are possible without departing from the spirit and scope of the invention.

1. A microtube for surgery and dentistry, the microtube comprising:
a tubular member having anterior and posterior ends, a side port, an axial opening at the posterior end of the tubular member, and an inner optical core of a material capable of transmitting, guiding, and directing a laser beam, the inner optical core extending from the axial opening at the posterior end of the tubular member through the side port.

2. The microtube of claim 1, wherein the optical core includes an optical fiber.

3. The microtube of claim 1, wherein the anterior end of the tubular member is conically tapered from wide to narrow in a direction away from the posterior end toward the anterior end of the tubular member.

4. A method for a surgical or dental procedure, the method comprising the steps of:
(a) providing a microtube comprising a tubular member having anterior and posterior ends, a side port, an axial opening at the posterior end of the tubular member, and an inner optical core of a material capable of transmitting, guiding, and directing a laser beam, the inner optical core extending from the axial opening at the posterior end of the tubular member through the side port; (b) disposing the side port of the tubular member at the site of the surgical or dental procedure; (c) disposing the axial opening at the posterior end of the tubular member at a source of the laser beam; and (d) delivering the laser beam through the side port to the site of the surgical or dental procedure, thereby combining mechanical and chemical debridement into a single procedure, enabling removal of pulpal tissue in three-dimensional volume elements which files and other instruments cannot reach, sterilizing the site of the surgical or dental procedure, and ablating the pulpal tissue.

5. The method of claim 4, wherein the optical core includes an optical fiber.

6. The method of claim 4, wherein the anterior end of the tubular member is conically tapered from wide to narrow in a direction away from the posterior end toward the anterior end of the tubular member.

7. A microtube for surgery and dentistry, the microtube comprising:
a tubular member having anterior and posterior ends, a side port, and an axial opening at the posterior end of the posterior end of the tubular member; and
means for transmitting, guiding, and directing a laser beam through the side port.
